(19) United States
(12) Patent Application Publication
(10) Pub. No.: US 2004/0016062 A1
(43) Pub. Date: Jan. 29, 2004

(54) Nondyeing composition containing a precursor and an oxidation reaction catalyst

(75) Inventor: Gregory Plos, Paris (FR)

Correspondence Address: STEPTOE & JOHNSON LLP, 1330 Connecticut Avenue, NW, Washington, DC, 20036-1795, US

(73) Assignee: L'OREAL, Paris (FR)

(21) Appl. No.: 10/374,713

(22) Filed: Feb. 27, 2003

Related U.S. Application Data
(60) Provisional application No. 60/382,592, filed on May 24, 2002.

(30) Foreign Application Priority Data
Mar. 1, 2002 (FR) 0202652

(51) Int. Cl. A61K 7/13

(52) U.S. Cl. 8/405

(57) ABSTRACT

The invention relates to a nondyeing cosmetic composition intended for lasting shaping or for texturization of keratinous materials containing, in a cosmetically acceptable medium, at least one compound as agent capable of oxidative polymerization and at least one agent catalyzing this oxidative polymerization reaction, and a method for treating keratinous materials with said cosmetic composition.

The present application relates to a nondyeing cosmetic composition intended for permanently shaping keratinous materials and for their texturization, and to a method using such a composition.

Many people have difficulties in giving their locks the shape which they desire. That is in particular the case for people with locks of fine hair. Fine hair strands are not sufficiently rigid to allow the hair-style, once in place, to be able to last over time. The hairstyle, subjected to movement and to moisture and to adverse weather conditions, loses its shape quite rapidly. This phenomenon is particularly pronounced in the case of locks of fine hair, but is also a problem in the case of locks of medium and thick hair.

Treatments have long been sought which allow the hairstyles to last longer over time.

Products exist which are based on the use of polymers.

These products are provided in the form of lacquers, sprays, gels and mousses. These lacquers and the sprays have a powerful effect, but give the hair-style an artificial appearance and feel because bonding is created between the hair strands.

Gels and mousses, when used in combination with blow drying, offer better resistance of the shape, while leaving a fairly natural appearance and feel. However, the effect is quite moderate and in many cases, in particular for fine hair strands, is considered to be insufficient.

Another technique is based on the modification of the disulfide bonds in the hair. The so-called “permanent-waving” products used to this end generally contain thiols or sulfites, which are capable of “reducing” the disulfide bridges in keratin which is the essential component of hair and thus “embrittle” the structure of the hair. During this time, being subjected to a mechanical stress (it is, for example curled), the hair takes a new shape. At the end of the treatment, the disulfide bridges of the hair being reduced, they have to be reoxidized in order to reestablish the cohesion of the hair. This method causes degradation of the keratinous fibers. Moreover, the sensitivity of the fibers thus treated to subsequent dyeings and bleachings is modified. Another disadvantage is that the method is quite long because it requires several steps: rolling up, application of a first product, leaving in, rinsing, application of a second product, leaving in, rinsing, unrolling, rinsing and final washing. In addition, these methods are fairly uncomfortable, in particular because of the odor of the first liquid, the odor released during the first leaving in and the residual odor left in the hair.

However, these methods are advantageous since their effect is lasting and is resistant to shampoos.

Advantageously and unexpectedly, the applicant has found a composition which does not exhibit the disadvantages of the prior art compositions, while leading to lasting shaping of keratinous fibers and in particular of hair.

Indeed, this composition comprises an agent capable of oxidative polymerization inside the fiber. Consequently, the use of these compositions does not confer an artificial appearance and feel on the hair-style.

These compositions allow application in a single step which is considerably shorter in duration than the use of conventional permanent waving.

Furthermore, the compositions according to the invention have the advantage of conferring, after washing and drying, a notable body effect, that is to say an effect which results in the sensation, to the touch, that the hair is thicker. This notion is particularly perceptible on fine hair strands. The expression texturization of hair is then used.

Because of the products used and the duration of application, the hair undergoes less impairment than when it is treated by a conventional permanent waving method.

The invention makes it possible to obtain, even on fine hair, a notable styling effect, an improved retention over time with, in particular, resistance to several shampoos, and a glossy effect which is difficult to obtain with commercial products.

Accordingly, a subject of the present application relates to a nondyeing cosmetic composition, this composition containing, in a cosmetically acceptable medium, at least one compound as agent capable of oxidative polymerization and at least one agent catalyzing this oxidative polymerization reaction.

Preferably, the agent capable of oxidative polymerization is a heterocycle condensed with a benzene ring. Still more preferably, this agent is an indole or an indoline.

The expression nondyeing composition is understood to mean, according to the present invention, a composition which, when applied to the hair, causes a modification of the colorimetric factor “AE” for hair following treatment of less than 5 units, and preferably less then 2 units.

The colorimetric factor “ΔE” is defined by the so-called L*a*b* method, also called “CIELAB methods” in the following manner:

ΔE=[(L* final-L* initial)²+(a* final-a* initial)² +(b* final-b* initial)²]^{1/2}

in which L* represents the luminance, a* the color on the green-red axis and b* represents the color on the blue-yellow axis.

This colorimetric factor is measured with the aid of a calorimeter such as the MINOLTA CM2002 colori-meter. The error on the measurement “ΔE” is very low.

The agent capable of oxidative polymerization which can be used for the purposes of the present patent application is preferably an agent capable of combining with itself to form a polymer containing at least 2 units. Preferably, this polymer will comprise between 2 and 10 000 units.

The preferred agents capable of polymerization are methyl 5,6-dihydroxyindoline-1-isopropionate and 1-hydroxyethyl-4-hydroxyindole.

The compositions according to the invention preferably contain from 0.001% to 20%, and still more preferably from 0.05% to 10% by weight of agent(s) capable of oxidative polymerization relative to the total weight of said composition.

The expression “cosmetically acceptable medium”, for the purposes of the present application, is under-stood to mean an aqueous medium or a medium containing one or more solvents or alternatively a medium consisting of a mixture of water and of one or more solvents.

The solvents may be chosen in particular from monoalcohols, and preferably lower alcohols such as ethanol, polyols such as propylene glycol, glycerin, polyethylene glycols, ethers or esters of these mono-alcohols or polyols.

The oxidative polymerization is preferably carried out using atmospheric oxygen as oxidizing agent.

The agents catalyzing the oxidation reaction or catalysts which can be used for the purposes of the present invention are in particular metals and their cosmetically acceptable salts and oxidative enzymes.

In particular, manganese, iron, copper, cobalt and their cosmetically acceptable salts, such as the sulfates, will be used as catalyst.

The oxidative enzymes which may be used are:

oxidoreductases containing 4 electrons, such as laccases and tyrosinases,

oxidoreductases containing 2 electrons, which catalyze the oxidation of a substrate by air in order to form an oxidized species capable of inducing the oxidation of the agent capable of polymerization. They are in particular glucose oxidase or uricase.

In the case where the cosmetic composition according to the invention comprises hydrogen peroxide, the enzyme peroxidase may be used as agent catalyzing the oxidative polymerization reaction. The hydrogen peroxide may be generated in situ by the enzymatic route.

In a preferred embodiment of the invention, the catalyst is a laccase.

The compositions according to the invention preferably contain from 0.00001% to 5% and still more preferably from 0.001% to 1% by weight of oxidation catalyst(s) relative to the total weight of said composition.

According to a particular variant of the invention, the cosmetic composition comprises, in addition, at least one oxidation reaction mediator. These mediators are intended to facilitate the oxidation reaction.

A particular embodiment of the invention consists in using i) an indole capable of oxidative polymerization, ii) an oxidation catalyst and iii) an oxidation mediator.

The enzymatic mediator(s) used in the composition in accordance with the invention make it possible to increase the enzymatic activity of the oxidoreductase used and may, for example, be chosen from the compounds of the following formula (I), and their possible tautomeric forms

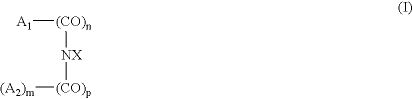

in which:

A₁ and A₂, which are identical or different, represent:

a) a linear or branched, saturated or unsatura- ted aliphatic radical containing from 1 to 30 carbon atoms, it being possible or otherwise for said aliphatic radical to be substituted with one or more hydroxyl, halogen, sulfo, carboxyl, nitro or phenyl radicals;

b) a heterocyclic radical comprising from 1 to 4 heteroatoms and from 5 to 10 members which may or may not be substituted with one or more C₁-C4 alkyl, halogen, phenyl, hydroxyl or C₇-C₁₀ aralkyl radicals;

c) an aromatic radical comprising from 6 to 10 members, it being possible or otherwise for said aromatic radical to be substituted with one or more C₁-C₄ alkyl, halogen, sulfo, carboxyl, nitro, hydroxyl or nitroso radicals;

it being possible for the nitrogen atom of the group NX to form, with the groups A₁-(CO)n and A₂-(CO)p, a heterocycle comprising from 5 to 18 members, it being possible or otherwise for said heterocycle to be substituted with one or more C₁-C₄ alkyl, hydroxyl, phenyl, halogen, sulfo, carboxyl or nitro radicals;

x represents a group −OH, =O, →O or →S;

m, n and p, independently of each other, are integers equal to 0 or 1.

Among the enzyme mediators of formula (I) above, there may be mentioned in particular hydroxylamine, N,N-dipropylhydroxylamine, N,N-diisopropylhydroxylamine, phenylhydroxylamine, N-acetylhydroxylamine, 1-phenyl-1H-1,2,3-triazole-l-oxide, 2,4,5-triphenyl-2H-1,2,3-tria-zole -1-oxide, 1-hydroxybenzotriazole, 1-hydroxybenzo-triazolesulfonic acid, 1-hydroxybenzimidazole, N-hydroxy-phthalimide, N-hydroxysuccinimide, quinoline N-oxide, isoquinoline N-oxide, 1-hydroxypiperidine, violuric acid, 4-hydroxy-3-nitrosocoumarin, 1,3-dimethyl-5-nitrosobarbituric acid, 1-nitroso-2-naphthol, 2-nitroso-1-naphthol-4-sulfonic acid, 2-nitroso-l-naphthol, 1-nitroso-2-naphthol-3,6-disulfonic acid, and 2,4-dinitroso-1,3-dihydroxybenzene.

The enzyme mediator(s) used in the ready-to-use composition in accordance with the invention may also be chosen from syringic acid and its esters, aceto-syringone; syringaldehyde; para-hydroxycinnamic acid; vanillin; 7-hydroxycoumarin; 2,4-dichlorophenol; para-hydroxybenzenesulfonate; 2,2′-azino-bis(3-ethyl-benzo-thiazoline -6-sulfonate); phenothiazines such as 10-methylphenothiazine; benzidines such as 3,3-dimethyl-benzidine; amino derivatives of 2-naphthalenesulfonic acid; L-tyrosine; ferulic acid; caffeic acid; chloro-genic acid and sinapic acid.

The compositions according to the invention preferably contain from 0.00001% to 5%, and still more preferably from 0.001% to 1% by weight of mediator relative to the total weight of said composition.

The cosmetic composition according to the present invention may also contain another agent chosen from the group consisting of nonionic, anionic, cationic or amphoteric surfactants, organic or inorganic thickeners, nonionic, anoinic, cationic or amphoteric polymers, exhibiting hair-styling properties or otherwise, linear or cyclic, volatile or nonvolatile silicones, which are organomodified or otherwise, vitamins or provitamins, and perfumes.

A second subject of the invention consists in the use of the nondyeing compositions according to the invention defined above, for lasting shaping of keratinous materials, and in particular of human keratinous fibers such as hair.

A third embodiment of the invention consists in the use of the nondyeing compositions according to the invention defined above, for texturing keratinous materials, and in particular human keratinous fibers such as hair.

The fourth subject of the present invention is also a method for treating keratinous materials, and in particular hair, such that a cosmetic composition according to the present invention is applied to these keratinous materials.

This method according to the present invention may be carried out on dry or wet hair. Preferably, application to wet hair will be preferred.

The duration of application of the composition according to the invention is between 10 seconds and 1 hour, preferably this application will last between 10 seconds and 30 minutes.

Preferably, after application of the composition, the keratinous materials are rinsed.

The method for treating keratinous materials according to the present invention may, in addition, be combined with one or more (2, 3, 4) other conventional treatments.

Accordingly, the treatment according to the present invention may be applied before, during or after these other treatments.

Accordingly, it is possible to carry out the permanent hair-styling method according to the present invention, before or during or after a conventional dyeing, bleaching or alternatively permanent waving, treatment.

In the case of a treatment in several steps, the treatment according to the present invention may take place between two steps of said conventional treatment.

The examples below illustrate the invention without limiting the scope thereof.

### EXAMPLE 1

The following composition is prepared:

| | |
|---|---|
| methyl 5,6-dihydroxyindoline- | 3 · 10⁻³ mol |
| 1-isopropionate (structure below) | |
| LACCASE | 0.015 g as proteins |
| | that is 400 000 units |
| phosphate buffer pH 7/ethanol (50/50) | qs 100 g |
| 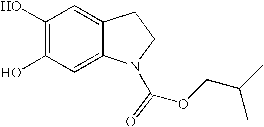 | |

The activity, in laccase units, is, at a given pH (here 7) the quantity of enzymes which causes, at 25° C., modification of absorbance from 0.001 to 435 nm from a 27 mM solution of para-phenylenediamine for an optical path length of 1 cm.

The composition is applied to natural gray hair. After leaving in for 30 minutes, the hair is rinsed. It is observed that the hair remains practically gray (colorimetric factor ΔE of 3.76 points).

Compared with untreated hair, the hair exhibits a notable body effect (texturization). This body effect is found after washing, and then drying the hair.

### EXAMPLE 2

The following composition is prepared:

| | |
|---|---|
| methyl 5,6-dihydroxyindoline- | 3 · 10⁻³ mol |
| 1-isopropionate (structure below) | |
| LACCASE | 0.015 g as proteins |
| | that is 400 000 units |
| violuric acid | 0.5 g |
| phosphate buffer pH 7/ethanol (50/50) | qs 100 g |
| 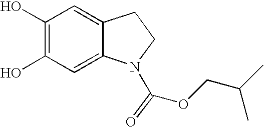 | |

The composition is applied to natural gray hair. After leaving in for. 30 minutes, the hair is rinsed. It is observed that the hair remains practically gray (colorimetric factor ΔE of 1.34 points).

Compared with untreated hair, the hair exhibits a marked body effect. This body effect is found after washing, and then drying the hair.

### EXAMPLE 3

The following composition is prepared:

| | |
|---|---|
| 1-hydroxyethyl-4-hydroxyindole | 0.01 g |
| (structure given below) | |
| LACCASE | 0.015 g as proteins |
| | that is 400 000 units |
| phosphate buffer pH 7/ethanol (70/30) | qs 100 g |
| 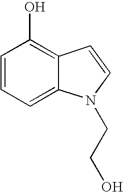 | |

The composition is applied to natural gray hair. After leaving in for 30 minutes, the hair is rinsed. It is observed that the hair remains practically gray (colorimetric factor ΔE of 1.9 points).

Compared with untreated hair, the hair exhibits a notable body effect. This body effect is found after washing, and then drying the hair.

1. A nondyeing cosmetic composition containing, in a cosmetically acceptable medium, at least one compound as agent capable of oxidative polymerization and at least one agent catalyzing this oxidative poly-merization reaction.

2. The cosmetic composition as claimed in claim 1, wherein the agent capable of oxidative poly-merization is a heterocycle condensed with a benzene ring.

3. The cosmetic composition as claimed in claim 2, wherein the agent capable of oxidative poly-merization is an indole or an indoline.

4. The cosmetic composition as claimed in any one of the preceding claims, wherein the catalyst falls within the group consisting of metals and their cosmetically acceptable salts, and oxidative enzymes.

5. The cosmetic composition as claimed in claim 4, wherein the catalyst falls within the group consisting. of manganese, iron, copper, cobalt and their cosmetically acceptable salts.

6. The cosmetic composition as claimed in claim 4, wherein the catalyst falls within the group consisting of the following enzymes: oxidoreductases containing 2 electrons, and oxidoreductases containing 4 electrons.

7. The cosmetic composition as claimed in claim 6, wherein the catalyst is a laccase.

8. The cosmetic composition as claimed in one of claims 1 to 3, which contains hydrogen peroxide or a peroxidase as catalyst.

9. The cosmetic composition as claimed in one of the preceding claims, wherein the agent capable of polymerization is chosen from methyl 5,6-dihydroxy-indoline -l-isopropionate and 1-hydroxyethyl-4-hydroxy-indole.

10. The cosmetic composition as claimed in one of the preceding claims, which preferably contains from 0.001% to 20%, and still more preferably from 0.05% to 10% by weight of agent(s) capable of oxidative poly-merization relative to the total weight of said composition.

11. The cosmetic composition as claimed in one of the preceding claims, which preferably contains from 0.00001% to 5%, and still more preferably from 0.001% to 1% by weight of oxidation catalyst(s) relative to the total weight of said composition.

12. The cosmetic composition as claimed in one of the preceding claims, which comprises at least one oxidation reaction mediator.

13. The cosmetic composition as claimed in one of the preceding claims, which comprises at least one other agent chosen from the group consisting of nonionic, anionic, cationic or amphoteric surfactants, organic or inorganic thickeners, nonionic, anoinic, cationic or amphoteric polymers, exhibiting hair-styling properties or otherwise, linear or cyclic, volatile or nonvolatile silicones, which are organomodified or otherwise, vitamins or provitamins, and perfumes.

14. The method for treating keratinous materials, wherein a cosmetic composition as claimed in one of claims 1 to 13 is applied to these keratinous materials for a period of between 10 seconds and one hour, preferably between 10 seconds and 30 minutes.

15. The method of treatment as claimed in claim 14, wherein the application of the cosmetic composition is followed by a step of rinsing.

16. The method of treatment as claimed in claim 14 or 15, wherein the method is combined with at least one other treatment chosen from conventional dyeings, bleachings and permanent wavings.

17. The use of a composition as claimed in one of claims 1 to 13, for the lasting shaping of keratinous materials, and in particular human keratinous fibers such as hair.

18. The use of a composition as claimed in claims 1 to 13, for texturing keratinous materials, and in particular human keratinous fibers such as hair.
